(19) United States
(12) Patent Application Publication
(10) Pub. No.: US 2004/0137472 A1
(43) Pub. Date: Jul. 15, 2004

(54) Methods and compositions for modification of splicing of pre-mRNA

(76) Inventor: Ryszard Kole, Chapel Hill, NC (US)

Correspondence Address: MYERS BIGEL SIBLEY & SAJOVEC, PO BOX 37428, RALEIGH, NC, 27627, US

(21) Appl. No.: 10/672,501

(22) Filed: Sep. 26, 2003

Related U.S. Application Data
(60) Provisional application No. 60/414,141, filed on Sep. 27, 2002.

(51) Int. Cl. C12Q 1/68; C12P 19/34

(52) U.S. Cl. 435/6; 435/91.2

(57) ABSTRACT

The present invention provides a method of preventing a splicing event in a pre-mRNA molecule, comprising contacting the pre-mRNA and/or elements of the splicing machinery with a small molecule compound identified according to the methods described herein to prevent the splicing event in the pre-mRNA molecule. Further provided is a method of inducing a splicing event in a pre-mRNA molecule, comprising contacting the pre-mRNA and/or elements of the splicing machinery with a small molecule compound identified according to the methods described herein to induce the splicing event in the pre-mRNA molecule. Furthermore, a method is provided herein of treating a patient having a disorder associated with an alternative or aberrant splicing event in a pre-mRNA molecule, comprising administering to the patient a therapeutically effective amount of a compound identified according to the methods described herein to prevent an alternative or aberrant splicing event in a pre-mRNA molecule, thereby treating the patient.

### RELATED APPLICATIONS

This application claims priority under 35 U.S.C. § 119(e) to U.S. provisional application No. 60/414,141, filed Sep. 27, 2002, the entire contents of which are incorporated by reference herein.

### FIELD OF THE INVENTION

The present invention relates to methods of modifying splicing events in pre-mRNA molecules and treating disorders associated with alternative or aberrant splicing events, as well as compounds and compositions useful in carrying out the methods of this invention.

### BACKGROUND OF THE INVENTION

U.S. Pat. No. 5,976,879 to Kole et al. describes antisense oligonucleotides, which inhibit aberrant and restore correct splicing or modify alternative splicing.

### SUMMARY OF THE INVENTION

The present invention provides a method of identifying a compound capable of modulating (e.g., preventing or inducing) a splicing event in a pre-mRNA molecule, comprising: a) contacting the compound with i) a cDNA comprising a disruption by an intron that renders the cDNA incapable of being expressed to produce a gene product in the absence of modulation of a splicing event, and ii) elements of the splicing machinery; and b) detecting expression of the cDNA to produce a gene product, thereby identifying a compound capable of modulating a splicing event in a pre-mRNA molecule.

Further provided is a method of treating a subject having a disorder associated with an alternate or aberrant splicing event in a pre-mRNA molecule, comprising administering to the subject a therapeutically effective amount of a compound identified according to the methods herein.

Additionally provided is a method of upregulating expression of a native protein in a cell containing a DNA encoding the native protein, wherein the DNA contains a mutation that causes downregulation of the native protein by aberrant splicing thereof, comprising introducing into the cell a compound identified according to the methods herein, whereby the aberrant splicing is inhibited, thereby resulting in upregulation of the native protein.

Furthermore, the present invention provides a method of upregulating expression of an alternative protein in a cell containing a DNA encoding the alternative protein, wherein the DNA is controlled by a first splicing event that results in downregulation of the alternative protein, comprising introducing into the cell a compound identified by the method of claim 1 to modulate splicing whereby the first splicing event is inhibited and a second splicing event occurs, thereby upregulating expression of the alternative protein.

In further embodiments, the present invention provides a composition comprising a compound identified by the methods of this invention and a pharmaceutically acceptable carrier.

The present invention also provides, in one aspect, a method of preventing or inducing a splicing event in a pre-mRNA molecule comprising contacting the pre-mRNA or other elements of the splicing machinery with a compound identified according to the methods described herein to prevent or induce the splicing event in the pre-mRNA molecule. Also provided by this invention is a method of identifying a compound capable of modulating (i.e., preventing or inducing) a splicing event in a pre-mRNA molecule comprising contacting the compound with cells as described in the Examples herein and/or with elements of the splicing machinery as described herein under conditions whereby a positive (prevention or induction of splicing) or negative (no prevention or no induction of splicing) effect is produced and detected an identifying a compound that produces a positive effect as a compound capable of preventing or inducing a splicing event.

In another aspect, the present invention provides a method of treating a patient having a disorder associated with an alternative or aberrant splicing event in a pre-mRNA molecule, comprising administering to the patient in a pharmaceutically acceptable carrier a therapeutically effective amount of a compound identified according to the methods described herein to prevent or induce an alternative or aberrant splicing event in a pre-mRNA molecule, thereby treating the patient.

The foregoing and other objects and aspects of the present invention are explained in detail in the specification set forth below.

### BRIEF DESCRIPTION OF THE DRAWINGS

FIG. 1 illustrates an EGFP construct that contains EGFP cDNA interrupted with intron 2 of the β-globin gene. Two point mutations at position 654 or at position 705 within intron 2 each create an aberrant 5′ splice site and activate the same cryptic 3′ splice site. These splice sites are preferentially utilized even though the correct splice sites still exist. As a result of the aberrant splicing pattern (shown on the right), a portion of intron 2 is retained in the spliced EGFP mRNA. This prevents functional EGFP protein production and no green fluorescence is produced. However, if correct splicing (shown on the left) can be restored through the use of antisense oligonucleotides or the use of small molecules, the intron is completely spliced out and a functional EGFP protein can be generated. The small arrows in bottom panel indicate primers used for RT-PCR.

FIG. 2 shows examples of cells treated with positive compounds. Panel A: Bright fluorescence of IVS2-705U EGFP cells treated with compound BB2. Panel B: Autofluorescing compound with extremely low levels of fluorescence of IVS2-705U EGFP cells treated with compound H4. Panel C: Low levels of fluorescence of IVS2-654 EGFP cells treated with compound F8. Panel D: Spots of compound autofluorescence with fluorescence of IVS2-654 EGFP cells treated with compound C11. Panel E: The positive control cell line has an EGFP 654 construct with a compensatory mutation that restores correct splicing without use of antisense oligonucleotides or small compounds. These cells constitutively express GFP. Panel F: Untreated IVS2-705U EGFP cells. An Olympus inverted fluorescence microscope was used to detect fluorescence.

FIG. 3 shows RT-PCR analysis of RNA isolated from IVS2-654 EGFP cells or IVS2-705U EGFP cells treated with several positive compounds. An example of a gel with easily identifiable correction is provided. Negative control lane is untreated IVS2-654 EGFP cells.

FIG. 4 shows RT-PCR analysis of RNA isolated from IVS2-705 β-globin cells treated with compound DD2 that causes high levels of correction as compared to other positive compounds. Lanes 1 and 2 are untreated ISV2-705 β-globin cells.

FIG. 5 shows RT-PCR analysis of RNA isolated from IVS2-705 EGFP cells treated with DD2, which causes low levels of correction on IVS2-705 EGFP cells correction as compared to other positive compounds. Lanes 1 and 2 are untreated IVS2-705U EGFP cells.

FIG. 6 demonstrates that compound DD2 causes fluorescence on IVS2-705 EGFP and β-globin HeLa cells. Panel A: the positive control cell line has an EGFP 654 construct with a compensatory mutation that restores correct splicing without use of antisense oligonucleotides or small compounds. These cells constitutively express GFP. Panel B: the negative control is untreated IVS2-705U EGFP cells. Panel C: IVS2-705U EGFP cells treated with 50 μM DD2. Panel D: IVS2-705 β-globin cells treated with 50 μM DD2. An Olympus inverted microscope was used to detect fluorescence.

FIG. 7 demonstrates that compound DD2 causes dose-dependent correction of splicing in IVS2-705 β-globin cells. The cells were treated with compounds and were incubated for 24 hours. RNA was isolated and an RT-PCR assay was performed. Correction of IVS2-705 β-globin cells by free uptake of PNA-2 antisense oligonucleotides served as a control.

### DETAILED DESCRIPTION OF THE INVENTION

Introns are portions of eukaryotic DNA, which intervene between the coding portions, or “exons,” of that DNA. Introns and exons are transcribed into RNA termed “primary transcript, precursor to mRNA” (or “pre-mRNA”). Introns must be removed from the pre-mRNA so that the native protein encoded by the exons can be produced (the term “native protein” as used herein refers to naturally occurring, wild type, or functional protein). The removal of introns from pre-mRNA and subsequent joining of the exons is carried out in the splicing process.

The splicing process is a series of reactions, which are carried out on RNA after transcription but before translation and which are mediated by splicing factors. Thus, a “pre-mRNA” is an RNA that contains both exons and intron(s), and an “mRNA” is an RNA in which the intron(s) have been removed and the exons joined together sequentially so that the protein may be translated therefrom by the ribosomes.

Introns are defined by a set of “splice elements” that are part of the splicing machinery and are required for splicing and which are relatively short, conserved RNA segments that bind the various splicing factors, which carry out the splicing reactions. Thus, each intron is defined by a 5′ splice site, a 3′ splice site, and a branch point situated therebetween. Splice elements also comprise exon splicing enhancers and silencers, situated in exons, and intron splicing enhancers and silencers situated in introns at a distance from the splice sites and branch points. In addition to splice site and branch points these elements control alternative aberrant and constitutive splicing.

The present invention provides the unexpected discovery that certain small chemical molecules can modify specific splicing events in specific pre-mRNA molecules. These small molecules can operate by a variety of mechanisms to modify the splicing event. For example, the small molecules of this invention can: 1) interfere with the formation and/or function and/or other properties of splicing complexes, spliceosomes, and their components such as hnRNPs, snRNPs, SR-proteins and other splicing factors or elements, resulting in the prevention or induction of a splicing event in a pre-mRNA molecule. As another example, 2) the small molecules of1 this invention can prevent and/or modify transcription of gene products, which can include, for example, but are not limited to, hnRNPs, snRNPs, SR-proteins and other splicing factors, which are subsequently involved in the formation and/or function of a particular spliceosome. 3). The small molecules of this invention can also prevent and/or modify phosphorylation, glycosylation and/or other modifications of gene products, including but not limited to, hnRNPs, snRNPs, SR-proteins and other splicing factors, which are subsequently involved in the formation and/or function of a particular spliceosome. 4) Additionally, the small molecules of this invention can bind to and/or otherwise affect specific pre-mRNA so that a specific splicing event is prevented or induced via a mechanism that does not involve basepairing with RNA in a sequence-specific manner. Thus, the small molecules of this invention are different from and are not related to antisense or antigene oligonucleotides.

The modulation of splicing events by the compounds of this invention includes modulation of naturally occurring alternative or alternate splicing and can include restoration of the correct or desired splicing event and also includes prevention of aberrant splicing events caused by mutations that can cause or are associated with genetic disease.

In embodiments wherein prevention of aberrant splicing is desired, the mutation in the native DNA and pre-mRNA can be either a substitution mutation or a deletion mutation that creates a new, aberrant, splice element. The aberrant splice element is thus one member of a set of aberrant splice elements that define an aberrant intron. The remaining members of the aberrant set of splice elements can also be members of the set of splice elements that define the native intron. For example, if the mutation creates a new, aberrant 3′ splice site which is both upstream from (i.e., 5′ to) the native 3′ splice site and downstream from (i.e., 3′ to) the native branch point, then the native 5′ splice site and the native branch point may serve as members of both the native set of splice elements and the aberrant set of splice elements. In other situations, the mutation may cause native regions of the RNA that are normally dormant, or play no role as splicing elements, to become activated and serve as splicing elements. Such elements are referred to as “cryptic” elements.

For example, if the mutation creates a new aberrant mutated 3′ splice site which is situated between the native 3′ splice site and the native branch point, it may activate a cryptic branch point between the aberrant mutated 3′ splice site and the native branch point. In other situations, a mutation may create an additional, aberrant 5′ splice site which is situated between the native branch point and the native 5′ splice site and may further activate a cryptic 3′ splice site and a cryptic branch point sequentially upstream from the aberrant mutated 5′ splice site. In this situation, the native intron becomes divided into two aberrant introns, with a new exon situated therebetween.

Further, in some situations where a native splice element (particularly a branch point) is also a member of the set of aberrant splice elements, it can be possible to block the native element and activate a cryptic element (i.e., a cryptic branch point) which will recruit the remaining members of the native set of splice elements to force correct splicing over incorrect splicing. Note further that, when a cryptic splice element is activated, it may be situated in either the intron or one of the adjacent exons. Thus, depending on the set of aberrant splice elements created by the particular mutation, a compound of this invention can block a variety of different splice elements to carry out the instant invention: it may block a mutated element, a cryptic element, or a native element; it may block a 5′ splice site, a 3′ splice site, or a branch point. In general, it will not block a splice element which also defines the native intron, of course taking into account the situation where blocking a native splice element activates a cryptic element which then serves as a surrogate member of the native set of splice elements and participates in correct splicing, as discussed above.

In other embodiments, an alternate splicing event can be modulated by employing the compounds of this invention. For example, a compound of this invention can be introduced into a cell in which a gene is present that comprises alternate splice sites. In the absence of the compound, a first splicing event occurs to produce a gene product having a particular function. In the presence of the compound of this invention, the first splicing event is inhibited and a second or alternate splicing event occurs, resulting in expression of the same gene to produce a gene product having a different function. Compounds having the ability to modulate alternate splicing events are identified according to the methods of this invention.

Thus, in specific embodiments, the present invention provides a method of preventing or inducing a splicing event in a pre-mRNA molecule, comprising contacting the pre-mRNA molecule and/or other elements of the splicing machinery (e.g., within a cell) with a compound identified according to the methods described herein to prevent or induce the splicing event in the pre-mRNA molecule. The splicing event that is prevented or induced can be either an aberrant splicing event or an alternate splicing event.

Further provided herein is a method of identifying a compound capable of preventing or inducing a splicing event in a pre-mRNA molecule, comprising contacting the compound with splicing elements and/or factors involved in alternative, aberrant and/or constitutive splicing as described herein (e.g., within cells as described in Example 1) under conditions whereby a positive (prevention or induction of splicing) or negative (no prevention or induction of splicing) effect is produced and detected and identifying a compound that produces a positive effect as a compound capable of preventing or inducing a splicing event.

In a further embodiment, the present invention provides compositions comprising a compound identified according to the methods described herein to prevent or induce an alternative or aberrant splicing event in a pre-mRNA molecule, in a pharmaceutically acceptable carrier. As noted above, the compounds of the present invention are not antisense or antigene oligonucleotides. Table 5 shows the chemical structure of several compounds of this invention as examples of the compounds of this invention and is not intended to be all-inclusive. Additional compounds are contemplated to be part of the present invention that have been or can be identified to have the characteristics described herein according to the protocols provided herein.

In another embodiment, the present invention provides a method of upregulating expression of a native protein in a cell containing a DNA encoding the native protein, wherein the DNA contains a mutation that causes downregulation of the native protein by aberrant and/or alternate splicing thereof. More particularly, the DNA encodes a pre-mRNA having the characteristics described herein. The method comprises introducing into the cell a small molecule of this invention that has been identified as described herein as a compound that prevents an aberrant splicing event, whereby the native intron is removed by correct splicing and the native protein is produced by the cell. In further embodiments, the present invention provides methods comprising introducing into a cell a small molecule of this invention that has been identified to modulate an alternate splicing event to produce a protein that has a different function than the protein that would be produced without modulation of alternate splicing.

The present invention also provides means for using the compounds of this invention to upregulate expression of a DNA containing a mutation that would otherwise lead to downregulation of that gene by aberrant splicing of the pre-mRNA it encodes. Accordingly, in one embodiment, the present invention provides a method of preventing aberrant splicing in a pre-mRNA molecule containing a mutation and/or preventing an alternate splicing event. When present in the pre-mRNA, the mutation causes the pre-mRNA to splice incorrectly and produce an aberrant mRNA or mRNA fragment different from the mRNA ordinarily resulting from the pre-mRNA. More particularly, the pre-mRNA molecule contains: (i) a first set of splice elements defining a native intron which is removed by splicing when the mutation is absent to produce a first mRNA molecule encoding a native protein, and (ii) a second set of splice elements induced by the mutation which define an aberrant intron different from the native intron, which aberrant intron is removed by splicing when the mutation is present to produce an aberrant second mRNA molecule different from the first mRNA molecule. The method comprises contacting the pre-mRNA molecule and/or other factors and/or elements of the splicing machinery as described herein (e.g., within a cell) with a compound identified by the methods described herein to prevent an aberrant splicing event in a pre-mRNA molecule, whereby the native intron is removed by correct splicing and the native protein is produced by the cell.

Further provided is a method of upregulating expression of a DNA that would otherwise be downregulated by modulating an alternate splicing event in the DNA. The method comprises contacting the pre-mRNA molecule and/or other elements and/or factors of the splicing machinery as described herein (e.g., within a cell) with a compound of this invention identified to modulate alternate splicing events, whereby a regular splicing event is inhibited and an alternate splicing event occurs to allow upregulated expression of a DNA that is otherwise downregulated when under the control of the regular splicing event.

The methods, compounds and compositions of the present invention have a variety of uses. For example, they are useful in any process where it is desired to have a means for downregulating expression of a gene to be expressed until a certain time, after which it is desired to upregulate gene expression (e.g., downregulate during the growth phase of a fermentation and upregulate during the production phase of the fermentation). For such use, the gene to be expressed may be any gene encoding a protein to be produced so long as the gene contains a native intron. The gene may then be mutated by any suitable means, such as site-specific mutagenesis (see T. Kunkel, U.S. Pat. No. 4,873,192) to deliberately create an aberrant second set of splice elements which define an aberrant intron which substantially downregulates expression of the gene. The gene may then be inserted into a suitable expression vector and the expression vector inserted into a host cell (e.g., a eukaryotic cell such as a yeast, insect, or mammalian cell (e.g., human, rat)) by standard recombinant techniques. The host cell is then grown in culture by standard techniques. When it is desired to upregulate expression of the mutated gene, a suitable compound of the present invention, in a suitable formulation, is then added to the culture medium so that expression of the gene is upregulated.

The methods, compounds and formulations of the present invention are also useful as in vitro or in vivo tools to examine and modulate splicing events in human or animal genes, which are developmentally, and/or tissue regulated (e.g., alternate splicing events). Such experiments may be carried out by the procedures described herein below, or modification thereof, which will be apparent to skilled persons.

The compounds and formulations of the present invention are also useful as therapeutic agents in the treatment of disease involving aberrant and/or alternate splicing, such as β-thalassemia (wherein the oligonucleotide would bind to β-globin, particularly human, pre-mRNA), (α-thalassemia (wherein the oligonucleotide would bind to α-globin pre-mRNA), Tay-Sachs syndrome (wherein the oligonucleotide would bind to β-hexoseaminidase α-subunit pre-mRNA), phenylketonuria (wherein the oligonucleotide would bind to phenylalanine hydroxylase pre-mRNA) and certain forms of cystic fibrosis (wherein the oligonucleotide would bind the cystic fibrosis gene pre-mRNA), in which mutations leading to aberrant splicing of pre-mRNA have been identified (See, e.g., S. Akli et al., J. Biol. Chem. 265, 7324 (1990); B. Dworniczak et al., Genomics 11, 242 (1991); L-C. Tsui, Trends in Genet. 8, 392 (1992)).

Examples of β-thalassemia which may be treated by the present invention include, but are not limited to, those of the β¹¹⁰, IVS1⁶, IVS1⁶, IVS2⁶⁵⁴, IVS2⁷⁰⁵, and IVS2⁷⁴⁵ mutant class (i.e., wherein the β-globin pre-mRNA carries the aforesaid mutations).

Other disorders associated with an alternative or aberrant splicing event in a pre-mRNA molecule that call be treated by the methods of the present invention include, but are not limited to, viral and retroviral infections, cancer, cardiovascular diseases, metabolic diseases including but not limited to, diabetes, inflammatory diseases including but not limited to arthritis, obesity.

Thus, in a further embodiment, the present invention provides a method of treating a patient having a disorder associated with an alternative or aberrant splicing event in a pre-mRNA molecule, comprising administering to the patient a therapeutically effective amount of a compound identified according to the methods described herein to modulate an alternative splicing event or prevent an aberrant splicing event and restore a correct splicing event in a pre-mRNA molecule, in a pharmaceutically acceptable carrier, thereby treating the patient.

Formulations of the present invention comprise the small molecules of this invention in a physiologically or pharmaceutically acceptable carrier, such as an aqueous carrier. Thus, formulations for use in the present invention include, but are not limited to, those suitable for oral administration, parenteral administration, including subcutaneous, intradermal, intramuscular, intravenous and intraarterial administration, as well as topical administration (e.g., administration of an aerosolized formulation of respirable particles to the lungs of a patient afflicted with cystic fibrosis or lung cancer or a cream or lotion formulation for transdermal administration of patients with psoriasis). The formulations may conveniently be presented in unit dosage form and may be prepared by any of the methods well known in the art. The most suitable route of administration in any given case may depend upon the subject, the nature and severity of the condition being treated, and the particular active compound, which is being used, as would be readily determined by one of skill in the art.

The present invention also provides for the use of a compound of the present invention having the characteristics set forth above for the preparation of a medicament for upregulating gene expression in a patient having a disorder associated with aberrant or alternate splicing of a pre-mRNA molecule, as discussed above. In the manufacture of at medicament according to the invention, the compound is typically admixed with, inter alia, a pharmaceutically acceptable carrier. The carrier must, of course, be acceptable in the sense of being compatible with any other ingredients in the formulation and must not be deleterious to the patient. The carrier may be a solid or a liquid. One or more compounds may be incorporated in any combination in the formulation of the invention, which may be prepared by any of the well-known techniques of pharmacy consisting essentially of admixing the components, optionally including one or more accessory therapeutic ingredients.

Formulations of the present invention may comprise sterile aqueous and non-aqueous injection solutions of the active compound, which preparations are preferably isotonic with the blood of intended recipient and essentially pyrogen free. These preparations may contain anti-oxidants, buffers, bacteriostats and solutes, which render the formulation isotonic with the blood of the intended recipient. Aqueous and non-aqueous sterile suspensions may include suspending agents and thickening agents. The formulations may be presented in unit dose or multi-dose containers, for example, sealed ampoules and vials, and may be stored in a freeze-dried (lyophilized) condition requiring only the addition of the sterile liquid carrier, for example, saline or water-for-injection immediately prior to use.

In one formulation, the compounds of this invention may be contained within a lipid particle or vesicle, such as a liposome or microcrystal, which may be suitable for parenteral administration. The particles may be of any suitable structure, such as unilamellar or plurilamellar, so long as the compound is contained therein. Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. No. 4,880,635 to Janoff et al.; U.S. Pat. No. 4,906,477 to Kurono et al.; U.S. Pat. No. 4,911,928 to Wallach; U.S. Pat. No. 4,917,951 to Wallach; U.S. Pat. No. 4,920,016 to Allen et al.; U.S. Pat. No. 4,921,757 to Wheatley et al.; etc.

The dosage of the compound administered will depend upon the particular method being carried out, when it is being administered to a subject, the disease, the condition of the subject, the particular formulation, the route of administration, etc. For administration to a subject such as a human, a dosage of from about 0.001, 0.01, 0.1, or 1.0 mg/Kg up to about 50, 100 or 150 mg/Kg is employed.

The examples, which follow, are set forth to illustrate the present invention, and are not to be construed as limiting thereof.

### EXAMPLE 1

### Library Screen

A commerically available of 10,000 small, drug-like molecules were screened for the ability to restore correct splicing patterns in the β-globin gene. The molecules ranged in molecular weight from 200 to 500 kD, were structurally diverse, and were pre-selected to have pharmacophore properties.

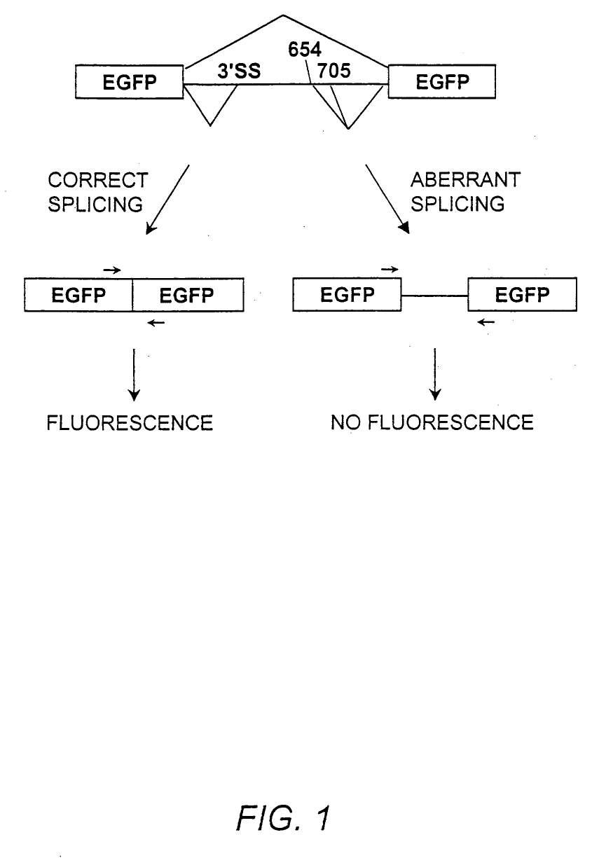

The screen was carried out using HeLa cell lines stably transfected with an EGFP (enhanced green fluorescent protein) construct. This construct contains the EGFP cDNA disrupted by intron 2 of the β-globin gene, containing thalassemic mutations (FIG. 1), and was under the control of the CMV promoter. Two different cell lines were generated with this construct, one with a mutation at position 654 (IVS2-654 EGFP) and another with a mutation at position 705 (IVS2-705U EGFP).

β-globin cell lines were obtained by stable transfection of HeLa cells with the human β-globin gene carrying thalassemic mutations at either IVS2-654 or IVS2-705. The cloned genes were under the control of the immediate early cytomegalovirus promoter (Sierakowska, et al., (1996) Proc. Natl. Acad. Sci. USA 93:12840-12844). The IVS2-654 EGFP cell line is well known in the art (Sazani (2001) Nucl. Acids Res. 29:3965-3974). The IVS2-705U EGFP cell line was made in the same manner using a different plasmid. HeLa cells were grown in SMEM with 5% horse serum, 5% fetal calf serum, 1% L-glutamine and 1% gentamicin/kanamycin.

For both constructs, disruption of EGFP with mutated intron 2 results in a lack of production of a functional EGFP protein. When correct splicing is restored to a cell, intron 2 is excised and functional EGFP protein is generated (Sazani (2001) Nucl. Acids Res. 29:3965-3974). This results in a fluorescent signal that is easily detectable by fluorescence microscopy.

Cells were plated in black well, clear bottom 96-well plates at 13×10⁴ cells/ml, 100 μl per well. The rows on the ends of the plate (rows 1 and 12) were control rows and the remaining rows were treated with compounds. In the control rows, positive control EGFP cells were alternated with untreated cells serving as negative controls. A cell line that contains an IVS2-654 EGFP construct with a compensatory mutation that restores correct splicing without use of antisense oligonucleotides or small compounds served as a positive control. Untreated IVS2-705U EGFP cells or IVS2-654 EGFP cells served as negative controls.

The compounds were a Prime Collection (Chembridge Corporation, San Diego, Calif.) consisting of a randomized library of 10,000 small molecules and a description of the compounds of the entire library and their production is incorporated herein in its entirety. The compounds were diluted in 20 μl DMSO with an approximate molecular weight of ˜500 g/mol, thus a ˜10 mM concentration. Compounds were further diluted 1:100 to a concentration of 100 μM in SMEM medium containing no serum, only 1% L-glutamine and 1% gentamicin/kanamycin.

Twenty-four hours after plating, HeLa medium was removed from the cells and 75 μl of medium containing 2× serum was added. (SMEM with 10% fetal calf serum, 10% horse serum, 1% L-glutamine and 1% gentamicin/kanamycin) Subsequently, 75 μl of diluted compounds was added to each well, bringing the amount of compound added to each well to a final concentration of 50 μM and the amount of serum in the medium to 1×.

Twenty-four hours after treatment, the medium was removed and the cells were washed 2 times in 150 μl 1× PBS. Cells were then fixed using 100 μl of 2% paraformaldehyde (PFA). The PFA was left on the cells 5-10 minutes and cells were again washed 2 times in 1× PBS leaving 150 μl of PBS in the wells after the final wash. Cells were then examined on an Olympus inverted fluorescence microscope at 10× or 20× using a FITC filter. Fluorescence was detected by eye. Attempts to utilize a fluorescence plate reader failed as the plate reader was not sensitive enough to detect the expected low levels of EGFP fluorescence. Treated cells were compared to the untreated cells to determine if the compounds caused the cells to fluoresce, which would indicate that the compound was correcting splicing.

The criteria used to designate a compound as a positive were as follows. The cells had to remain healthy after treatment, indicating that the compound was not toxic even at the relatively high concentration tested. Additionally, the fluorescence had to be mostly uniform throughout all of the cells. This criterion was complicated by the fact that many of the compounds had varying levels of autofluorescence, and therefore were difficult to distinguish from those that caused cellular fluorescence. Wells that contained large amounts of autofluorescing compound in strands, specks, or clumps were also considered potential positives.

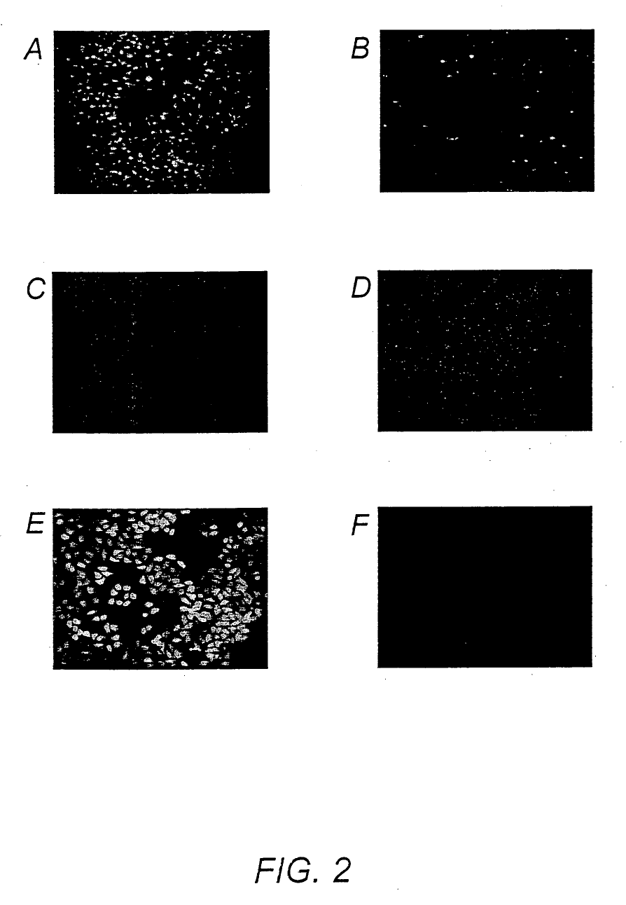

The initial screen provided 132 total positives (compounds that cause green fluorescence) from the 10,000 compounds tested on either IVS2-654 or IVS2-705U EGFP cells. Examples of cellular fluorescence in the presence of positive compounds are provided in FIG. 2. Compounds which provided fairly bright cellular fluorescence (FIG. 2, Panel A), autofluoresces with low levels of cellular fluorescence (FIG. 2, Panel B), and only low levels of cellular fluorescence (FIG. 2, Panel C) were detectable.

Of the 132 positive compounds, 13 caused fluorescence only on IVS2-654 EGFP cells and 16 were specific for IVS2-705U EGFP cells. The other 103 positives caused fluorescence on both cell lines. Of the 132 total positives, only about 15% were recorded as emitting a moderate or higher level of brightness with a uniform distribution. Another 20% emitted a low level of fluorescence, and 30% were noted as extremely faint. The remaining 35% appeared patchy or had some specks of compound autofluorescence.

The IVS2-705U and IVS2-654 EGFP cell lines were treated a second time with the 132 positive compounds and analyzed on a fluorescence microscope. It was noted that some compounds initially identified as positives did not emit a detectable fluorescent signal in the second examination. Additionally, some treated wells were filled with specks of autofluorescing compound.

### EXAMPLE 2

### RT-PCR Analysis of EGFP Positive Compounds

A RT-PCR-based assay was performed to determine if the fluorescence observed on the EGFP cells was a result of the compounds shifting splicing. This assay demonstrates that the aberrantly spliced product is slightly longer than the correctly spliced product because it contains a portion of intron 2. Therefore, primers located in exons 2 and 3 are used to PCR amplify the products, and the size difference is detected by running the products on an acrylamide gel.

After the IVS2-705U and IVS2-654 EGFP cells were re-treated with positive compounds and examined on the microscope, total RNA was isolated using TRI-REAGENT® (Molecular Research Center, Inc., Cincinnati, Ohio). A total of 200 ng of RNA was analyzed by reverse transcription polymerase chain reaction (RT-PCR) using rTth DNA polymerase (Perkin-Elmer, Boston, Mass.) according to manufacturer's instructions. The reverse transcription step was carried out at 70° C. for 15 minutes. The PCR step included 1 cycle, 95° C., 3 minutes: 18 cycles. 95° C., 1 minute, 65° C., 1 minute. For β-globin mRNA amplification, the reverse primer spanned positions 6-28 of exon 3 and the forward primer spanned positions 21-43 of exon 2 of the human β-globin gene. For EGFP mRNA amplification, the reverse primer was 5°-GTGGTGCAGATGAACTTCAGGGTC-3′ (SEQ ID No:1) and the forward primer was 5′-CGTAAACGGCCACAAGTTCAGCG-3′ (SEQ ID NO:2). RT-PCR products were separated on an 8% non-denaturing polyacrylamide gel and were visualized by autoradiography.

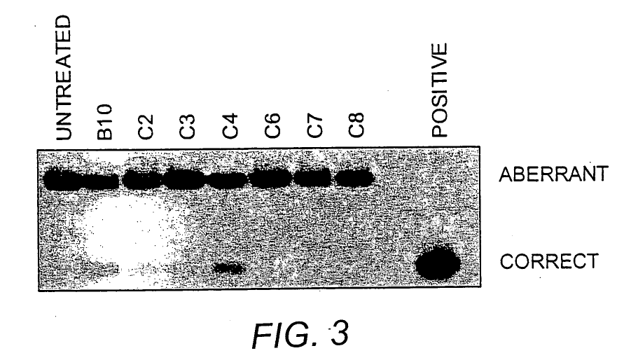

FIG. 3 provides an example of a polyacrylamide gel with RT-PCR products from EGFP cells treated with several positive compounds. The amount of correction was determined by comparing the ratio of the aberrant band and the correct band. Aberrant bands in FIG. 3 are all of comparable intensity and the correct band is readily identifiable.

The compounds causing correction by RT-PCR on IVS2-705U EGFP cells are indicated by a plus sign in Table 1. The compounds causing correction on IVS2-654 EGFP cells are provided in Table 2. A total of 11 compounds caused correction of IVS2-705U EGFP cells by RT-PCR and 11 compounds caused correction of IVS2-654 EGFP cells. Of those compounds causing correction, only 2 caused correction on both EGFP cell lines.

TABLE I

| | 705U EGFP | 705U EGFP | 705 β-globin |
|---|---|---|---|
| Compound | Fluorescence* | RT-PCR^{#} | RT-PCR^{#} |
| A2 | + | | |
| A3 | + | | |
| A4 | + | | |
| A5 | +/− | | |
| A6 | +/− | + | |
| A7 | − | + | |
| A8 | − | | |
| A9 | + | | + |
| A10 | + | | |
| A11 | +/− | | |
| B2 | +/− | | |
| B3 | + | | |
| B4 | + | | |
| B5 | + | | |
| B6 | + | | |
| B7 | − | | |
| B8 | − | | |
| B9 | + | | |
| B10 | + | | |
| B11 | + | | |
| C2 | + | | |
| C3 | + | | |
| C4 | + | | |
| C5 | + | | |
| C6 | + | | |
| C7 | − | | |
| C8 | + | | |
| C9 | + | | |
| C10 | + | | + |
| C11 | + | | + |
| D2 | + | | |
| D3 | + | + | |
| D4 | + | | |
| D5 | + | | |
| D6 | +/− | | |
| D7 | +/− | | |
| D8 | + | | |
| D9 | + | | |
| D10 | + | + | + |
| D11 | + | + | + |
| E2 | + | + | |
| E3 | − | | |
| AA2 | − | | |
| AA3 | − | | |
| AA4 | +/− | | |
| AA5 | +/− | | |
| AA6 | +/− | | |
| AA7 | +/− | + | |
| AA8 | − | | |
| AA9 | − | | |
| AA10 | − | | |
| AA11 | − | | + |
| BB2 | + | | |
| BB3 | − | | |
| BB4 | +/− | | |
| BB5 | +/− | | |
| BB6 | − | | |
| BB7 | +/− | | |
| BB8 | + | | |
| BB9 | +/− | | |
| BB10 | + | | |
| BB11 | − | | |
| CC2 | − | | |
| CC3 | − | | |
| CC4 | +/− | | |
| CC5 | +/− | | + |
| CC6 | + | | |
| CC7 | +/− | | |
| CC8 | − | + | + |
| CC9 | + | | |
| CC10 | + | | |
| CC11 | − | | |
| DD2 | + | + | + |
| DD3 | +/− | | |
| DD4 | + | | + |
| DD5 | +/− | | |
| DD6 | + | | |
| DD7 | + | | |
| DD8 | + | | |
| DD9 | +/− | | |
| DD10 | + | | |
| DD11 | +/− | | |
| EE2 | − | | |
| EE3 | − | + | |
| D2 | + | | |
| D3 | + | | |
| D4 | + | | |
| D5 | +/− | | |
| D6 | +/− | | |
| D7 | − | | |
| D8 | − | | |
| D9 | + | | |
| D10 | + | | |
| D11 | +/− | | |
| E2 | +/− | | |
| E3 | + | | |
| E4 | + | | + |
| E5 | + | | |
| E6 | + | | |
| E7 | − | | |
| E8 | − | | |
| E9 | + | | |
| E10 | + | | |
| E11 | − | + | + |
| F2 | − | | |
| F3 | + | | |
| F4 | + | | |
| F5 | + | | |
| F6 | + | | |
| F7 | − | | + |
| F8 | + | | |
| F9 | + | | |
| F10 | + | | |
| F11 | + | | |
| G2 | + | | |
| G3 | + | | |
| G4 | + | | |
| G5 | + | | |
| G6 | +/− | | |
| G7 | +/− | | |
| G8 | + | | |
| G9 | +/− | | |
| G10 | + | | |
| G11 | + | | |
| H2 | +/− | | |
| H3 | + | | |
| H4 | − | | |
| H5 | + | | |
| H6 | + | | |
| H7 | + | | |
| H8 | − | | |
| H9 | +/− | | |

*Compounds causing moderate to high levels of fluorescence with no autofluorescence of the compound itself are indicated by a (+) sign. A (+/−) indicates compounds causing faint levels of fluorescence or compounds that have some autofluorescence in addition to causing the cells to fluoresce. A (−) sign indicates that the compounds did not cause detectable levels of fluorescence.
^{#}Compounds causing correction by RT-PCR. are indicated by a (+) sign.

These results indicate that the compounds can restore correct splicing in a sequence-specific manner. Correlations between the presence of fluorescence and the correction of splicing as determined by RT-PCR were difficult to obtain. However, in general, a compound that causes correction in the RT-PCR assay does not necessarily cause detectable fluorescence when analyzed on a fluorescence microscope.

### EXAMPLE 3

### Treatment of β-globin Cell Lines with Positive Compounds

Cell lines of therapeutic significance include the IVS2-705 and IVS2-654 β-globin cell lines. These β-globin cells have different exonic sequences from the EGFP cells, which allowed for further investigation into the sequence specificity of the compounds provided herein. The β-globin cells were treated with all 132 positive compounds as provided above and then examined on a fluorescence microscope. As the β-globin cells do not contain an EGFP construct, correction of splicing would not result in a fluorescent signal. Therefore, any fluorescence detected was likely attributable to autofluorescence of the compound. However, most β-globin cells showed levels of fluorescence equal to that of EGFP cells treated with the same compounds. This indicated that most of the fluorescence observed on EGFP cells was a result of autofluorescence of the compound rather than the compound correcting splicing. However, as noted above, autofluorescence of the compound does not preclude the restoration of correct splicing even though it may mask fluorescence caused by this correction. Thus, RT-PCR was conducted to elucidate which compounds were correcting splicing.

The RT-PCR assay was performed on total RNA isolated from IVS2-705 and IVS2-654 β-globin cells treated with the positive compounds. The RT-PCR results are provided in Table 1 and Table 2 for IVS2-705 and IVS2-654 β-globin cells, respectively. Thirteen compounds caused correction on IVS2-705 β-globin cells while only one compound caused correction on IVS2-654 p-globin cells. This same compound (compound CCS) was the only compound that caused correction on all four cell lines examined.

The fluorescence data and the RRT-PCR results indicate that there is no apparent correlation between the levels of fluorescence and the amount of correction, i.e.. bright fluorescence does not necessarily correspond with more correction. The amount of correction caused by the compounds may have been too low to be detected on a fluorescence microscope. Only one compound caused significantly high levels of correction by RT-PCR. Additionally, a number of the compounds autofluoresced, making it appear as though high levels of correction had occurred. If these same compounds caused any correction in the RT-PCR assay, the correct band would be weak compared to the deceivingly high levels of fluorescence observed. Furthermore, the majority of compounds that showed correction of splicing in the RT-PCR assay did cause fluorescence on both EGFP and β-globin cells.

### EXAMPLE 4

### Analysis of Compounds That Did Not Cause Fluorescence

As the amount of fluorescence does not correlate well with the amount of correction, some compounds, which actually shift splicing, may have been missed in the initial screen due to the absence of detectable fluorescence. As a control, a panel of 20 compounds that did not cause fluorescence on EGFP cells was randomly chosen. Both IVS2-705U EGFP and IVS2-705 β-globin cells were treated with the negative compounds and examined on a fluorescence microscope. As in the initial screen, none of these compounds caused fluorescence on IVS2-705U EGFP cells. Furthermore, fluorescence was not detected on IVS2-705 β-globin cells. Thus, compounds that are negative for fluorescence on EGFP cells are also negative on globin cells.

RT-PCR analysis was performed on the IVS2-705U EGFP and IVS2-705 β-globin cells treated with the panel of negative compounds described above. These compounds did not cause fluorescence on either cell line, however some of the compounds did cause correction by RT-PCR on these cell lines. Two of the 20 compounds tested caused correction on IVS2-705 β-globin cells, while 5 of 20 caused correction on IVS2-705U EGFP cells. These results indicate that the EGFP screen used herein did not identify all compounds that could restore correct splicing.

### EXAMPLE 5

### Specificity of Compounds

As summarized in Table 3, the results provided in Table 1 indicate that some of the compounds correct splicing in both IVS2-705U EGFP and IVS2-705 β-globin cells while some compounds only correct splicing in one cell line or the other. Some compounds can shift splicing in both cell lines because the intronic sequences are virtually identical, except for a mutation at position 711 changing an A to a U in the IVS2-705U EGFP cells. However, the base at position 711 as well as the exonic sequences are different in the cell lines. Therefore, if these elements play important roles in whatever mechanism a particular compound uses to shift splicing, correction will be different between the two cell lines.

TABLE 3

| Correction by RT- | Correction by RT- | Compounds causing | Total Correct | Total Correct |
|---|---|---|---|---|
| PCR on 705U | PCR on | correction on both 705U | on 705U | on 705 |
| EGFP only | 705 β-globin only | EGFP& 705β-globin | EGFP | β-globin |
| 5* | 7^{#} | 5 | 11 | 13 |

*Excludes one compound that caused correction on both IVS2-705U EGFP and IVS2-654 EGFP cells.
^{#}Excludes one compound that caused correction on both IVS2-705 β-globin and IVS2-654 EGFP cells.

The results of RT-PCR analysis of RNA isolated from IVS2-654 EGFP and IVS2-654 β-globin cells treated with positive compounds are summarized in Table 4. The number of compounds causing correction on IVS2-654 EGFP cells is similar to the number that caused correction of IVS2-705 β-globin or IVS2-705U EGFP cells. In contrast, only one compound appeared to result in correction of IVS2-654 β-globin cells. These two IVS2-654 cell lines have identical β-globin intron 2 sequences.

TABLE 4

| Correction by | Correction by RT- | Compounds causing | | Total Correct |
|---|---|---|---|---|
| RT-PCR on 654 | PCR on | correction on both 654 | Total Correct | on 654 |
| EGFP only | 654 β-globin only | EGFP& 654 β-globin | on 654 EGFP | β-globin |
| 8* | 0 | 1 | 11 | 1 |

*Excludes one compound that caused correction on both IVS2-654 EGFP cells and 1VS2-705U EGFP or one compound that caused correction on both IVS2-654 EGFP cells and IVS2-705 β-globin.

Overall, the number of compounds causing correction of IVS2-654 EGFP and IVS2-705U EGFP was the same.

### EXAMPLE 6

### Compound DD2

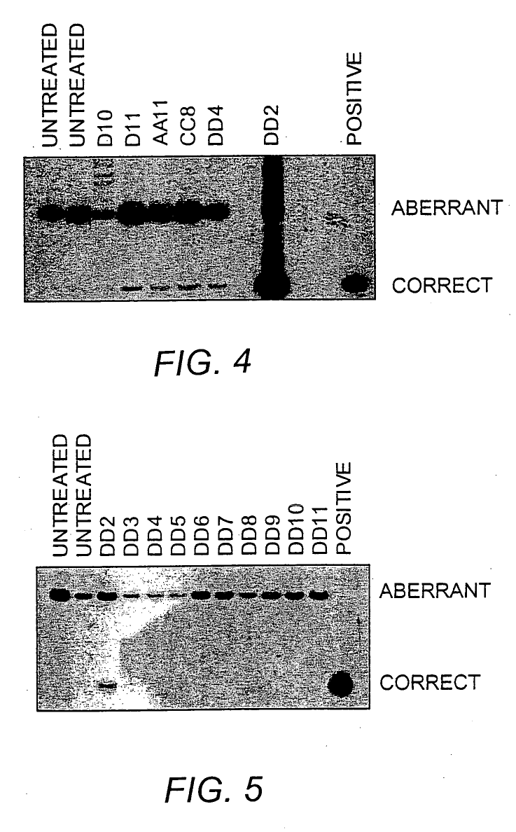

One compound resulted in virtually complete collection of splicing. This compound was designated DD2 and no other compound caused levels of correction as high as this lead compound. Several compounds previously identified as causing correction of IVS2-705 β-globin cells by RT-PCR were tested in a second RT-PCR with DD2 (FIG. 4).

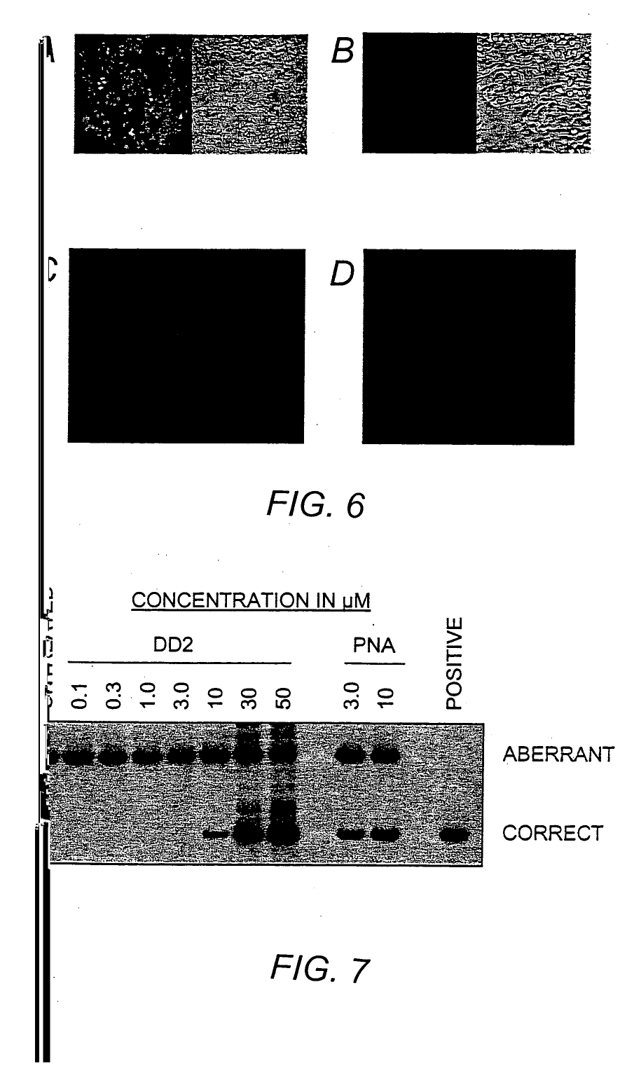

The results indicate that compound DD2 causes correction of IVS2-705 β-globin cells with high efficiency. Correction of IVS2-705U EGFP cells by DD2 occurs at a much lower level (FIG. 5). The reduced amounts of correction seen on the IVS2-705U EGFP cells may account for the fact that low levels of fluorescence are observed when EGFP cells are treated with DD2. If DD2 corrected splicing of IVS2-705U EGFP cells with the same efficiency with which it corrects splicing of IVS2-705 β-globin cells, bright fluorescence would be expected. However, the level of correction on IVS2-705U EGFP cells is potentially too low to cause significant fluorescence (FIG. 6). Compound DD2 also causes low levels of fluorescence on IVS2-705 β-globin cells, indicating the compound may have some autofluorescence.

### EXAMPLE 7

### DD2 Corrects Splicing in a Dose-Dependent Manner

IVS2-705 β-globin cells were treated with a range of concentrations of DD2. The concentrations tested were from 0.1 μM to 50 μM. The compound displayed dose-dependent correction of splicing (FIG. 7). The range of concentrations that caused correct splicing was relatively small; the lowest concentration that resulted in correction was 10 μM and approximately 20% correction was observed. A concentration of 30 μM caused about 60% correction while treatment with 50 μM resulted in approximately 85% correction of splicing (50 μM was the concentration used to treat cells in the initial screen). These results indicate that increasing the concentration of compound DD2 used to treat IVS2-705 β-globin leads to an increase in the amount of correct β-globin mRNA produced.

The foregoing examples are illustrative of the present invention, and are not to be construed as limiting thereof. The invention is described by the following claims, with equivalents of the claims to be included therein.

TABLE 5

| | |
|---|---|
| 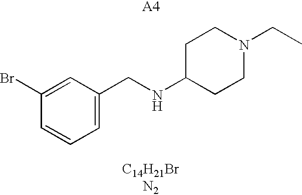 | 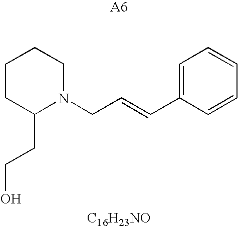 |
| 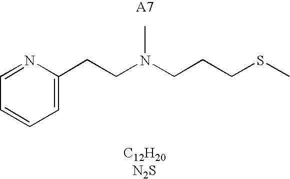 | 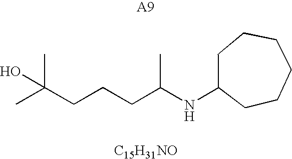 |
| 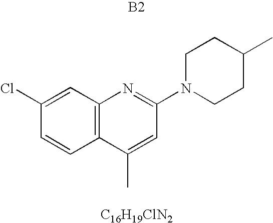 | 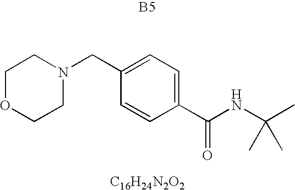 |
| 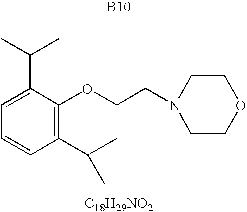 | 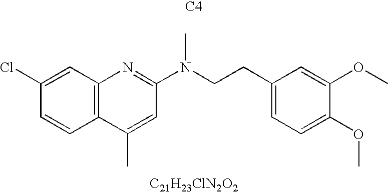 |
| 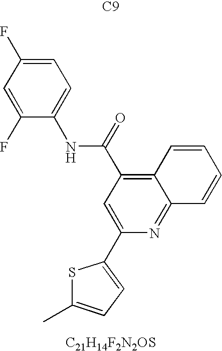 | 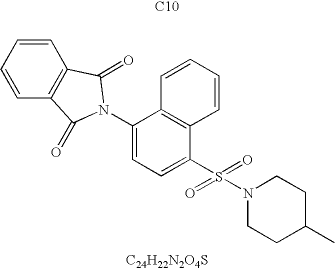 |
| 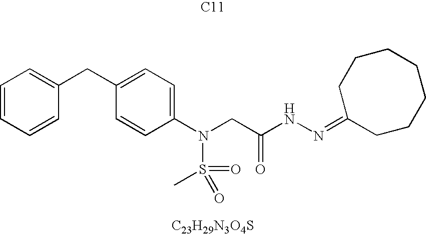 | 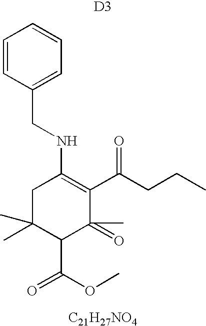 |
| 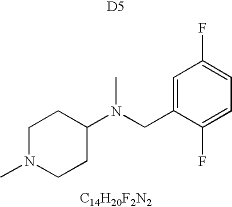 | 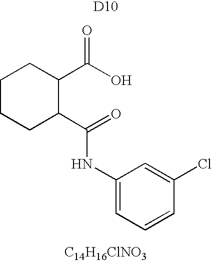 |
| 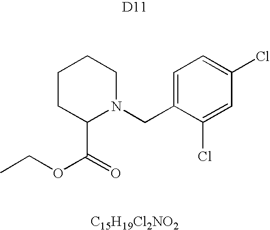 | 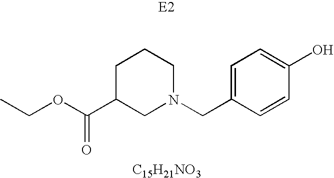 |
| 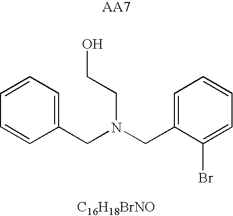 | 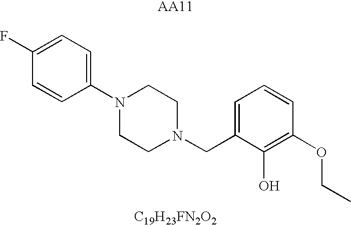 |
| 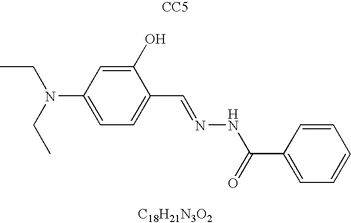 | 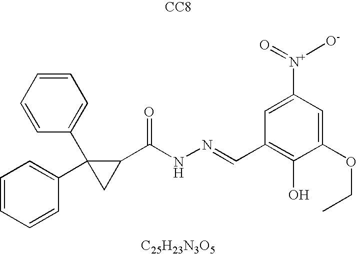 |
| 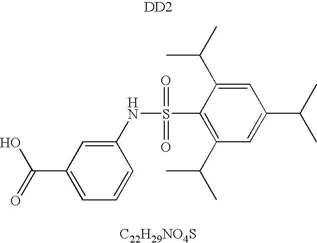 | 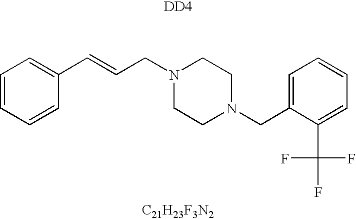 |
| 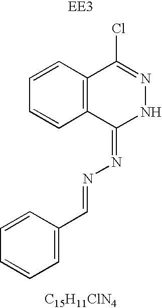 | 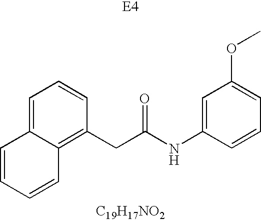 |
| 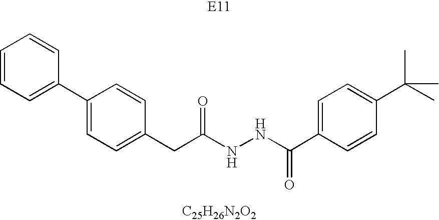 | 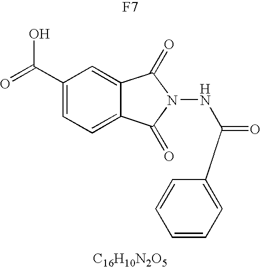 |

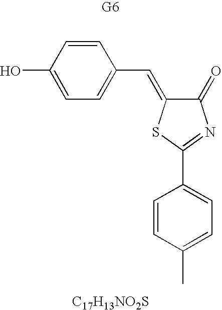

| | |
|---|---|
| 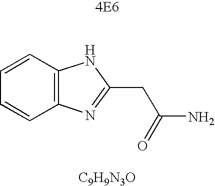 | 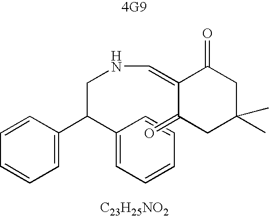 |

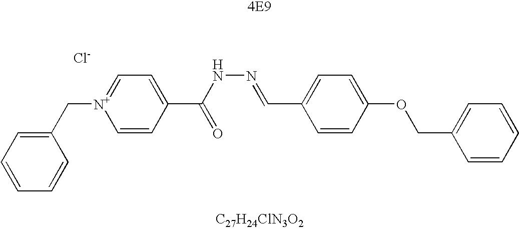

| | |
|---|---|
| 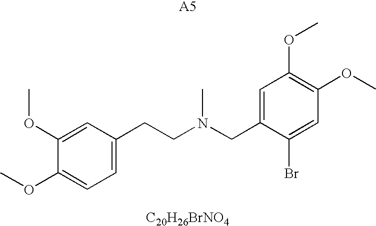 | 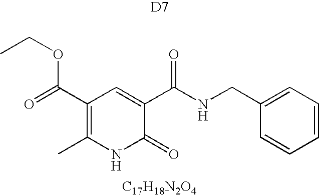 |
| 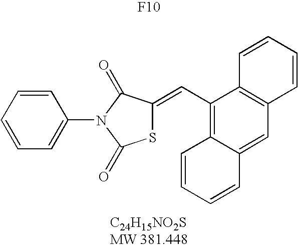 | 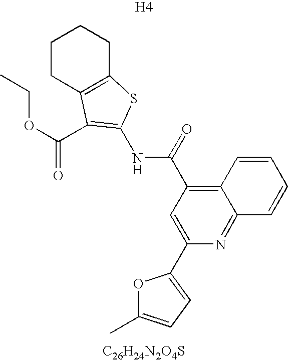 |
| 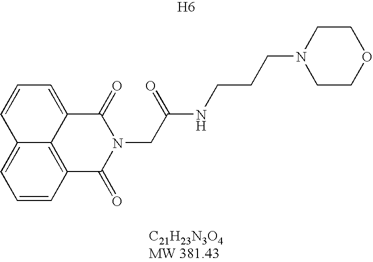 | 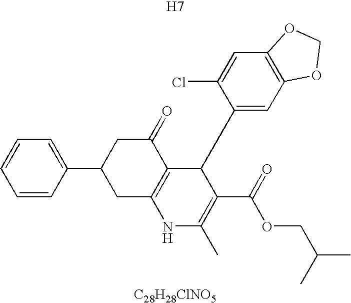 |

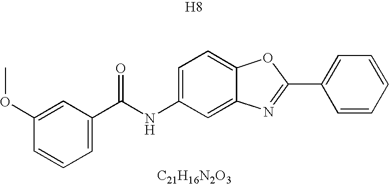

What is claimed is:

1. A method of identifying a compound capable of modulating a splicing event in a pre-mRNA molecule, comprising:
a) contacting the compound with a
i) a cDNA comprising a disruption by an intron that renders the cDNA incapable of being expressed to produce a gene product in the absence of modulation of a splicing event, and
ii) elements of the splicing machinery; and
b) detecting expression of the cDNA to produce a gene product, thereby identifying a compound capable of modulating a splicing event in a pre-mRNA molecule.

2. A method of preventing a splicing event in a pre-mRNA molecule in a cell, comprising contacting the cell with a compound identified according to the method of claim 1.

3. A method of inducing a splicing event in a pre-mRNA molecule in a cell, comprising contacting the cell with a compound identified according to the method of claim 1.

4. A method of treating a subject having a disorder associated with an alternate or aberrant splicing event in a pre-mRNA molecule, comprising administering to the subject a therapeutically effective amount of a compound identified according to the method of claim 1.

5. A method of upregulating expression of a native protein in a cell containing a DNA encoding the native protein, wherein the DNA contains a mutation that causes downregulation of the native protein by aberrant splicing thereof, comprising introducing into the cell a compound identified according to the method of claim 1, whereby the aberrant splicing is inhibited, thereby resulting in upregulation of the native protein.

6. A method of upregulating expression of an alternative protein in a cell containing a DNA encoding the alternative protein, wherein the DNA is controlled by a first splicing event that results in downregulation of the alternative protein, comprising introducing into the cell a compound identified by the method of claim 1 to modulate splicing whereby the first splicing event is inhibited and a second splicing event occurs, thereby upregulating expression of the alternative protein.

7. A composition comprising a compound identified by the method of claim 1 and a pharmaceutically acceptable carrier.
